(19) United States
(12) Patent Application Publication
(10) Pub. No.: US 2008/0213355 A1
(43) Pub. Date: Sep. 4, 2008

(54) Method and System for in Vivo Drug Delivery

(75) Inventor: Marcel Bohmer, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/995,851

(22) PCT Filed: Jul. 11, 2006
(86) PCT No.: PCT/IB06/52351

§ 371 Date: Jan. 16, 2008

Related U.S. Application Data
(60) Provisional application No. 60/701,633, filed on Jul. 22, 2005.

(51) Int. Cl. A61K 9/48; A61K 47/34; A61K 47/32; A61K 41/00

(52) U.S. Cl. 424/451; 514/772.7; 514/772.3; 514/772.4

(57) ABSTRACT

A system and method for polymeric drug delivery vehicles activated by ultrasound is disclosed herein. The system and method include polymeric particles, partially filled with a gas or a gas precursor, and partially filled with a liquid containing a drug. The drug is then released locally by application of ultrasound. Because the drug is dissolved, the delivery thereof is more efficient than for drugs incorporated with or in the polymeric shell of such particles.

The present disclosure relates generally to a therapeutic delivery system and method for targeted drug delivery. In particular, the present disclosure relates to a system and method for targeted drug delivery by combining a dissolved drug with a polymeric contrast agent and application of an ultrasound to release the drug encapsulated in a polymeric shell.

Targeted therapeutic delivery means are particularly important where the toxicity of a drug is an issue. Specific therapeutic delivery methods potentially serve to minimize toxic side effects, lower the required dosage amounts, and decrease costs for the patient. The present disclosure is directed to addressing these and/or other important needs in the area of therapeutic delivery.

Ultrasound is a diagnostic imaging technique, which is unlike nuclear medicine and X-rays since it does not expose the patient to the harmful effects of ionizing radiation. Moreover, unlike magnetic resonance imaging, ultrasound is relatively inexpensive and may be conducted as a portable examination. In using the ultrasound technique, sound is transmitted into a patient or animal via a transducer. When the sound waves propagate through the body, they encounter interfaces from tissues and fluids. Depending on the acoustic properties of the tissues and fluids in the body, the ultrasound sound waves are partially or wholly reflected or absorbed. When sound waves are reflected by an interface they are detected by the receiver in the transducer and processed to form an image. The acoustic properties of the tissues and fluids within the body determine the contrast, which appears in the resultant image.

Advances have been made in recent years in ultrasound technology. However, despite these various technological improvements, ultrasound is still an imperfect tool in a number of respects, particularly with regard to the imaging and detection of disease in the liver and spleen, kidneys, heart and vasculature, including measuring blood flow. The ability to detect and measure these regions depends on the difference in acoustic properties between tissues or fluids and the surrounding tissues or fluids. As a result, contrast agents have been sought which will increase the acoustic difference between tissues or fluids and the surrounding tissues or fluids in order to improve ultrasonic imaging and disease detection.

Changes in acoustic properties or acoustic impedance are most pronounced at interfaces of different substances with greatly differing density or acoustic impedance, particularly at the interface between solids, liquids and gases. When ultrasound waves encounter such interfaces, the changes in acoustic impedance result in a more intense reflection of the sound waves and a more intense signal in the ultrasound image. An additional factor affecting the efficiency or reflection of sound is the elasticity of the reflecting interface. The greater the elasticity of this interface, the more efficient the reflection of sound. Substances such as gas bubbles present highly elastic interfaces. Thus, as a result of the foregoing principles, researchers have focused on the development of ultrasound contrast agents based on gas bubbles or gas containing bodies and on the development of efficient methods for their preparation.

Currently, ultrasound contrast agents for medical diagnostics are typically gas bubbles encapsulated with a shell consisting of proteins, polymers or phospholipids or a combination thereof. Ultrasound imaging is based on the interaction of the contrast agent with the sound field, which can make use of the non-linear response of the contrast agent with techniques such as harmonic imaging and pulse inversion. Contrast agents containing fluorinated gases have been developed for this purpose.

Alternatively, contrast agents can be destroyed using a sound field. This is especially useful for polymeric agents with a rather stiff shell; upon liberation of the gas from the contrast agent, a short bright signal originates from a gas bubble, thus witnessing the destruction of the agent. As polymeric contrast agents usually have a thicker, less permeable shell than lipid shelled agents, fluorinated gases are often not used.

The destruction of the contrast agent can also be used to deliver therapeutic drugs at a specific location in the body. Such destruction can be established using ultrasound equipment designed for diagnostic purposes. The drug can be incorporated in the shell of the contrast agent, in a small particle attached to the contrast agent or in the interior of the contrast agent.

Experiments where the destruction of polymeric gas particles by ultrasound was followed by optical microscopy, showed that in many cases the particle shape does not change significantly after escape of the gas. Therefore, options to incorporate drugs into the shell material or on the shell are less preferred than drugs leaving the interior of the particle or capsule together with the escape of gas. For efficient local release, it is advantageous that the drug is already dissolved, especially for lipophilic drugs as disclosed in U.S. Pat. No. 6,416,740 to Unger et al., the contents of which are incorporated herein by reference in their entirety.

To date several different mechanisms have been developed to deliver therapeutic drugs to living cells using ultrasound. These mechanisms incorporate the drugs into the shell material or on the shell. These methods have not been proven in vivo. None of these methods potentiate local release, delivery and integration of the therapeutic drug to the target cell.

Better means of delivery for therapeutics are needed to treat a wide variety of human and animal diseases. Progress has been made in ultrasound drug delivery in vivo, however, a more efficient delivery is desired to obtain better dose control, better control over the energy needed to release the drugs and obtain longer circulation times for treatment of human and animal disease.

The present disclosure provides a system and method providing an effective polymeric drug delivery vehicle activated by ultrasound. In one embodiment, the system includes a capsule with a polymeric shell having two fluids inside, one fluid being an oil with a dissolved drug, the other fluid being a gas or liquid that can be phase converted to gas by ultrasound.

The present disclosure also provides a method for drug delivery making a capsule with a polymeric shell having two fluids inside one fluid being an oil with a dissolved drug, the other fluid being a gas or a liquid that can be phase converted to gas by ultrasound and delivery of the drug by exposing the capsules to ultrasound.

Additional features, functions and advantages associated with the disclosed system and method will be apparent from the detailed description, which follows, particularly when reviewed in conjunction with the figures appended hereto.

To assist those of ordinary skill in the art in making and using the disclosed system and method, reference is made to the appended figures, wherein:

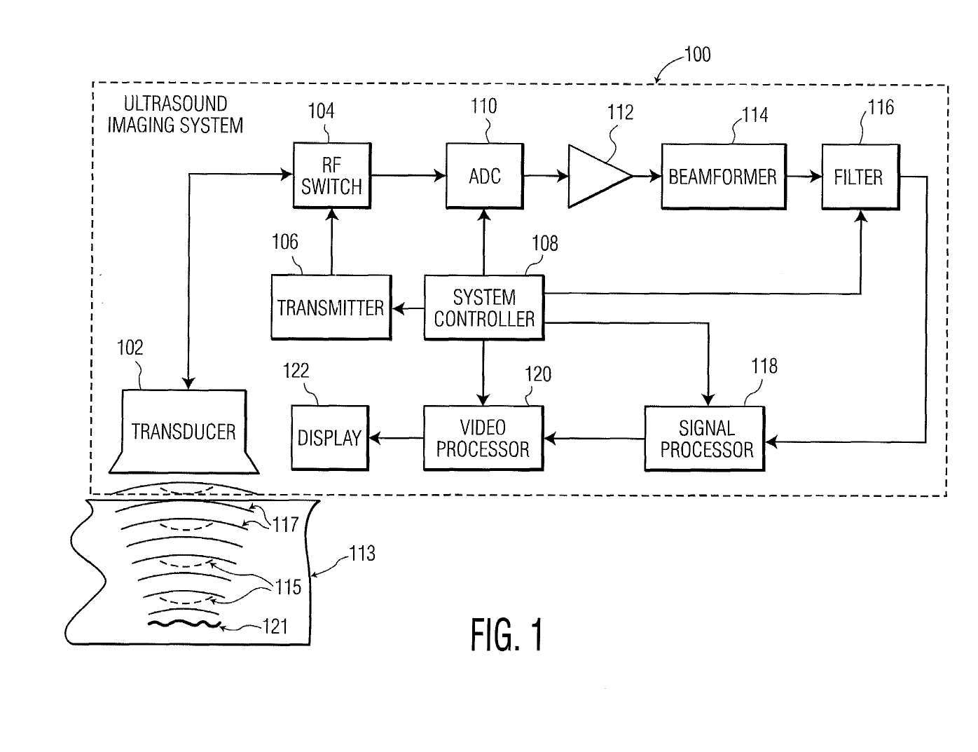

FIG. 1 is a block diagram of an ultrasonic imaging system consistent with the teachings of the present disclosure;

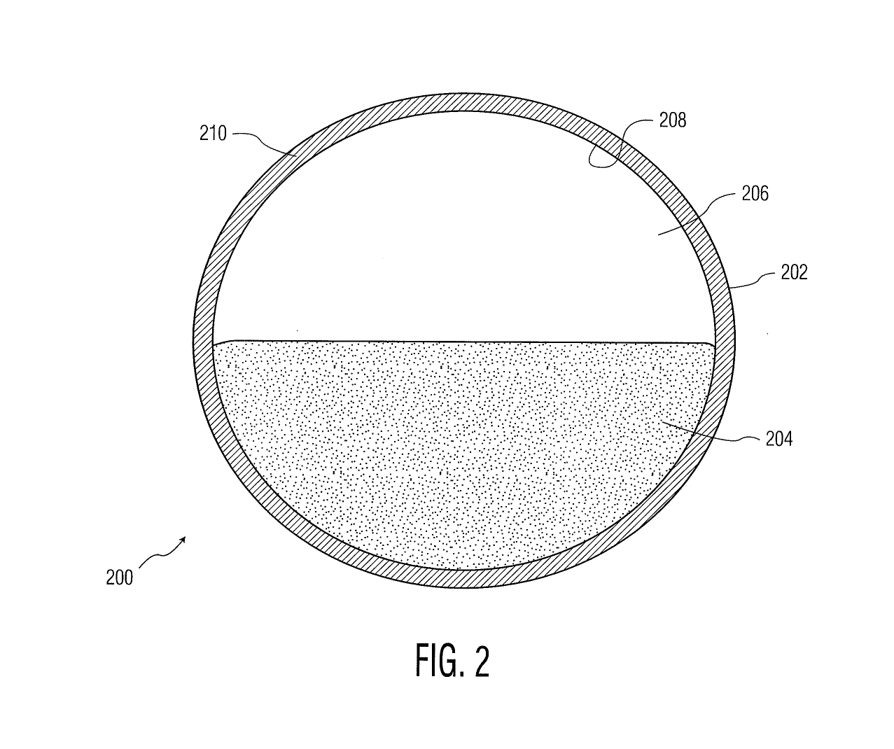

FIG. 2 is a cross sectional view of a polymer capsule partially filled with an oil containing a hydrophobic drug dissolved therewith and partially filled with a gas or liquid perfluorocarbon in accordance with an exemplary embodiment of the present disclosure; and

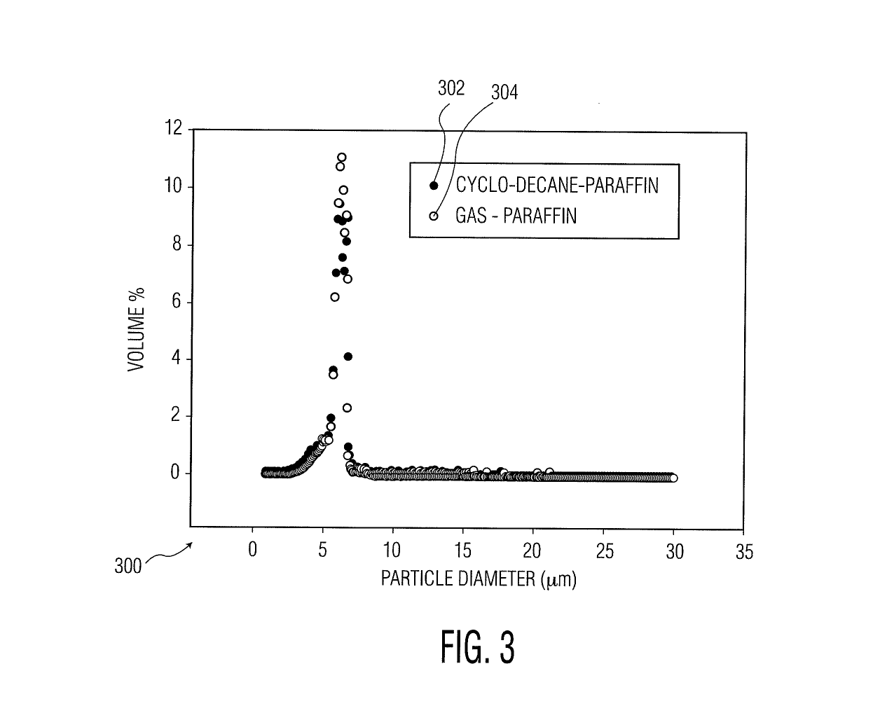

FIG. 3 is graph of particle size distribution of inkjetted capsules containing paraffin with a dissolved dye and cyclodecane before and after freeze drying in accordance with an exemplary embodiment.

As set forth herein, the system and method of the present disclosure advantageously permit and facilitate targeted drug delivery by encapsulating a dissolved drug with a polymeric contrast agent. Once the polymer capsule is introduced into the patient's body, a therapeutic compound may be targeted to specific tissues through the use of sonic energy causing the microspheres to rupture and release the therapeutic compound.

FIG. 1 depicts an ultrasound measuring and imaging system capable of viewing tissue and contrast agent(s) as may be adapted to and employed with an exemplary embodiment. In this regard, the ultrasound imaging system 100 may comprise a transducer 102, a RF switch 104, a transmitter 106, a system controller 108, an analog to digital converter (ADC) 110, a time gain control amplifier 112, a beamformer 114, a filter 116, a signal processor 118, a video processor 120, and a display 122. The transducer 102 may be electrically coupled to the RF switch 104. The RF switch 104 may be configured as shown with a transmit input coupled from the transmitter 106 and a transducer port electrically coupled to the transducer 102. The output of RF switch 104 may be electrically coupled to an ADC 110 before further processing by the time gain control amplifier 112. The time gain control amplifier 112 may be coupled to a beamformer 114. The beamformer 114 may be coupled to the filter 116. The filter 116 may be further coupled to a signal processor 118 before further processing in the video processor 120. The video processor 120 may then be configured to supply an input signal to a display 122. The system controller 108 may be coupled to the transmitter 106, the ADC 110, the filter 116, and both the signal processor 118 and the video processor 120 to provide necessary timing signals to each of the various devices.

As will be appreciated by persons having ordinary skill in the art, the system controller 108 and other processors, e.g., video processor 120 and signal processor 118, may include one or more processors, computers, and other hardware and software components for coordinating the overall operation of the ultrasonic imaging system 100. The RF switch 104 isolates the transmitter 106 of the ultrasound imaging system 100 from the ultrasonic response receiving and processing sections comprising the remaining elements illustrated in FIG. 1.

The system architecture illustrated in FIG. 1 provides an electronic transmit signal generated within the transmitter 106 that is converted to one or more ultrasonic pressure waves herein illustrated by ultrasound lines 115. When the ultrasound lines 115 encounter a tissue layer 113 that is receptive to ultrasound insonification the multiple transmit events or ultrasound lines 115 penetrate the tissue 113. As long as the magnitude of the multiple ultrasound lines 115 exceeds the attenuation affects of the tissue 113, the multiple ultrasound lines 115 will reach an internal target or tissue of interest 121, hereinafter referred to as tissue of interest. Those skilled in the art will appreciate that tissue boundaries or intersections between tissues with different ultrasonic impedances will develop ultrasonic responses at harmonics of the fundamental frequency of the multiple ultrasound lines 115.

As further illustrated in FIG. 1, such harmonic responses may be depicted by ultrasonic reflections 117. Those ultrasonic reflections 117 of a magnitude that exceed the attenuation effects from traversing tissue layer 113 may be monitored and converted into an electrical signal by the combination of the RF switch 104 and transducer 102. The electrical representation of the ultrasonic reflections 117 may be received at the ADC 110 where they are converted into a digital signal. The time gain control amplifier 112 coupled to the output of the ADC 110 may be configured to adjust amplification in relation to the total time a particular ultrasound line 115 needed to traverse the tissue layer 113. In this way, response signals from one or more tissues of interest 121 will be gain corrected so that ultrasonic reflections 117 generated from relatively shallow objects do not overwhelm in magnitude ultrasonic reflections 117 generated from insonified objects further removed from the transducer 102.

The output of the time gain control amplifier 112 may be beamformed, filtered and demodulated via beamformer 114, filter 116, and signal processor 118. The processed response signal may then be forwarded to the video processor 120. The video version of the response signal may then be forwarded to display 122 where the response signal image may be viewed. It will be further appreciated by those of ordinary skill in the art that the ultrasonic imaging system 100 may be configured to produce one or more images and or oscilloscopic traces along with other tabulated and or calculated information that would be useful to the operator.

Harmonic imaging can also be particularly effective when used in conjunction with contrast agents. In contrast agent imaging as discussed above, gas or fluid filled micro-sphere contrast agents known as microbubbles are typically injected into a medium, normally the bloodstream. Because of their strong nonlinear response characteristics when insonified at particular frequencies, contrast agent resonation can be easily detected by an ultrasound transducer. The power or mechanical index of the incident ultrasonic pressure wave directly affects the contrast agent acoustical response. At lower powers, microbubbles formed by encapsulating one or more gaseous contrast agents with a material forming a shell thereon resonate and emit harmonics of the transmitted frequency. The magnitude of these microbubble harmonics depends on the magnitude of the excitation signal pulse. At higher acoustical powers, microbubbles rupture and emit strong broadband signals.

The destruction of the contrast agent microbubbles can also be used to deliver drugs at a targeted location of a patient body. For efficient local release of the drug, it is advantageous that the drug is already dissolved. This is especially true for lipophilic drugs. See U.S. Pat. No. 6,416,740 to Unger et al., the content of which is incorporated herein by reference in its entirety. In the present disclosure, a dissolved drug is combined with a polymeric contrast agent rather than a lipid. The use of polymers advantageously allows obtaining longer circulation times and processing conditions can be chosen to obtain substantially narrow size distribution leading to better dose control of the drug.

It will be noted that the embodiments described herein can also be used in combination with focused ultrasound, for instance high intensity focused ultrasound (HIFU), devices which allow for the deposition of a higher amount of energy. More energy can be deposited using focused ultrasound or high intensity focused ultrasound to deliver drugs from particles, as higher intensities can be used, phase conversion of liquids can be achieved. Compared to bubbles that have a gaseous core at body temperature, these liquid filled particles have a much better lifetime in the circulation. For local drug delivery, it is desirable to have an agent that has a phase conversion above body temperature and below the boiling point of water. Perfluorocarbons have, compared to corresponding alkanes, relatively low boiling points. For example, perfluoro-octane has a boiling point of 99° C. and per-fluoro heptane has a boiling point of 80° C. If the heat of evaporation is low compared to that of water, cavitation can be achieved using ultrasound, especially with therapeutic ultrasound transducers. Having a boiling point above body temperature also leads to condensation once the ultrasound is stopped and the temperature in the region of interest (ROI) decreases again. As a result, the risk of formation of uncontrollable large gas bubbles is therefore minimized.

In one embodiment, the preparation of a polymeric contrast agent involves a freeze drying step in which a hollow core or microbubble is formed. The present disclosure proposes dissolving the drug in a solvent that cannot be removed by lyophilization (freeze drying) and adding a second liquid that can be removed by lyophilization. By using this combination, microbubble particles can be formed that have a core that is partially filled with liquid and partially filled with a gas. Then, application of ultrasound to the particles can rupture the microbubble cores releasing the drug.

In a second alternative embodiment, a particle containing two liquids can be used where one of the liquids can be phase converted using ultrasound, liquid perfluorocarbons like perfluorohexane, perfluorheptane, perfluorooctane, perfluorooctylbromide can be used for the second liquid as mentioned above. As these liquids do not have to be removed, the lyophilization step can be shortened or omitted.

Polymeric ultrasound contrast agents and drug delivery vehicles are made using emulsification methods. In an exemplary embodiment, a suitable polymer or a combination of polymers is dissolved in a solvent that is not miscible with water. Subsequently an emulsion is prepared. This emulsion can be further processed to remove the solvent, for instance by spraydrying as disclosed in U.S. Pat. No. 5,853,698 to Straub et al. and incorporated herein by reference in its entirety, or by extraction/evaporation of the solvent. At a certain stage in the processing the polymer will precipitate and form the shell. The latter process can be controlled more precisely by addition of a non-solvent for the polymer. This non-solvent controls the maximum shrinkage of the emulsion droplets, and therefore adds to the size control of the capsules. If the shrinkage of the emulsion droplets continues until all of the good solvent for the polymer has disappeared and all of the non-solvent is still present, optimum control over the shell thickness relative to the capsule diameter can be obtained.

In an exemplary embodiment, the non-solvent comprises a solvent that can be removed by lyophilization in combination with a non-solvent that is very hard to remove by lyophilization, thereby allowing a lipophilic drug to be dissolved in the oil phase (or: to remain dissolved in the oil phase after completion of the processing). For example, if the non-solvent comprises a solvent that can be removed by lyophilization, such as cyclooctane, cyclodecane, or dodecane, for example, in combination with a non-solvent that is very hard to remove by lyophilization, for example, paraffin or vegetable oils. It is also possible to use higher alkanes such as hexadecane. A lipophilic drug, such as deoxyrubicin or paclitaxel, can be dissolved in the oil phase.

FIG. 2 is a schematic representation of a liquid filled polymer capsule. The liquid filled capsule 200 includes a polymer shell 202 partially filled with an oil 204 containing a hydrophobic drug and partially filled with a second fluid 206 (e.g., gas or liquid). For example, second fluid 206 may include a gas or liquid perfluorocarbon, but is not limited thereto.

Suitable polymers for polymer shell 202 include synthetic biodegradable polymers such polylactides, polyglycolides, polycaprolactones, polycyanoacrylates and copolymers thereof. Biodegradeable polymers that can be used in the present disclosure are biopolymers, such as dextran and albumin or synthetic polymers such as poly(L-lactide acid) (PLA) and certain poly(meth)acrylates, polycaprolacton and polyglycolic-acid. Of particular interest are so-called (block) copolymers that combine the properties of both polymer blocks (e.g., hydrophobic and hydrophobic blocks). Examples of random copolymers are poly(L-lactic-glycolic acid) (PLGA) and poly(d-lactic-1-lactic acid) (Pd,1LA). Examples of diblock copolymers are poly(ethylene glycol)-poly(L-lactide) (PEG-PLLA), poly(ethylene glycol)-poly(N-isopropylacryl amide) (PEG-PNiPAAm) and poly(ethylene oxide)-poly(propylene glycol (PEO-PPO). An example of a triblock copolymer is poly(ethylene oxide)-poly(propylene glycol)-poly(ethyleneoxide) (PEO-PPO-PEO). Pegylation improves the circulation in the blood. Preferably, an inside surface 208 defining the inside of the capsule is hydrophobic to improve the gas retention in capsules made of the polymers enumerated above. This can be established by using a polymer with an alkyl or preferably a fluorinated end group as disclosed in U.S. Pat. No. 6,329,470 to Gardella, Jr. et al., the content of which is incorporated herein by reference in its entirety. Targeting moieties may be attached to an outside surface 210 defining the outside of the capsule 202.

Suitable or “good” solvents for these polymers and copolymers are relatively polar solvents such as dichloromethane, dichloroethane, isopropylacetate, acetone, and tetrahydrofuran, for example, but are not limited thereto. A production fluid is a solution of the constituting material, i.e. the material(s) of which the microspheres or polymer shells 202 are to be made in a solvent. In other words: the constituent(s) of the final microspheres are dissolved in a solvent. For example, in the solvent, polymer or monomers may be dissolved together with a non-solvent for the polymer and a drug. The solvent in the production fluid should have a limited solubility in the receiving fluid with the receiving fluid. The solvent will slowly diffuse into the receiving fluid and subsequently evaporate, leading to shrinkage of the drops of the production fluid. Good results are achieved at solubilities around 1%, such as is the case for dichloroethane (DCE) or dichloromethane (DCM) in water.

The continuous phase is aqueous and may contain polymeric stabilizers such as poly-vinyl alcohol (pva) or surfactants. If pegylated polymers are used, polymeric stabilizers are not always necessary.

Good maintenance of the size and distribution of the size of the microspheres is achieved when the micro-spheres form a stable colloid, which is facilitated by the presence of polymers or surfactant in the receiving fluid. The coalescence of droplets into larger droplets is then thereby counteracted/prevented. In a preferred embodiment, the production liquid contains a halogenated solvent which has a high density, such as DCE or DCM and the receiving solution is aqueous. Halogenated solvents with a small solubility in water (about 0.8% for dichloroethane) and a high vapor pressure are preferred for slow and controlled removal from the drops of production fluid. The constituents of the final microspheres are dissolved in the production fluid. For constituents to be used (intravenously) inside living humans, biodegradable polymers and (modified) phospholipids are preferred as carrier materials, drugs and imaging agents can be incorporated in the microspheres and targeted to markers of diseases expressed on blood vessel walls, such as markers for angiogenesis associated with tumors and markers for vulnerable plaques. After jetting, the excess stabilizer can be removed through a series of washing steps and the removal of the final remainders of the halogenated solvent can be established by lyophilization (freeze drying).

As the method outlined above leads to dense particles, it will also lead to dense shells, therefore giving a robust encapsulation of liquids or gases. To achieve this, the production liquid has to be modified with a non-solvent for the shell forming material.

In exemplary embodiments, emulsification may take place using mechanical agitation, extrusion through filters and other common means of emulsion preparation. For applications where particles with a well defined shell thickness and a narrow size distribution are required, drop-by-drop emulsification techniques such as inkjet printing, cross-flow emulsification and microchannel emulsification are preferred. In the above manner, an essentially monodisperse distribution of small sized microspheres is achieved, provided that the initial emulsion droplets are monodisperse. This can be achieved by jetting of the production fluid directly into the receiving fluid (e.g., without passing through air first) from a submerged nozzle. The manufacturing involves jetting of the production fluid at relatively high jetting rates, into a receiving fluid. It has been found that at low polymer concentrations in the production fluid, shrinkage of the droplet occurs providing essentially non-porous polymer microspheres.

These drop-by-drop emulsification techniques are especially preferred for the preparation of drug delivery vehicles that can be activated by ultrasound in accordance with exemplary embodiments of the present disclosure. The uniformity in the size and shell thickness provides excellent control over the amount of drug incorporated and the energy needed to open the shell encapsulating the drug for in vivo release.

After emulsification, the solvent is readily removed dichloromethane or dichloroethane is chosen, for example. Use can be made of the fact that these solvents have a limited solubility in water and that they have a high vapor pressure, as discussed above. Therefore, agitation thereof allows removal of these solvents from the emulsion. The solvents can also be removed by extraction. After disappearance of the solvent, liquid filled capsules 200 result, the liquid consisting of the non-solvent 206 for the polymer to be evaporated and the solvent 204 for the drug (FIG. 2). It will be recognized that the solvent 204 for the drug is preferably also a non-solvent for the polymer.

The capsules are then freeze-dried. In the case cyclo-octane is used, freeze drying can take place at a pressure of about 2 mbar. In the case of less volatile liquids to be removed, such as cyclo-decane or dodecane, the pressure is reduced to about 0.02 mbar, for example. These pressures are not sufficient to also remove oils like vegetable oil or paraffin, and therefore, the drug will stay dissolved in the oil or solvent 204.

FIG. 3 illustrates a size distribution 300 before and after freeze drying. More specifically, FIG. 3 illustrates a particle size distribution of inkjetted capsules 200 containing paraffin with a dissolved dye (e.g., oil blue N) and cyclo-decane (filled symbols) 302, for example. After freeze drying, the cyclo-decane is removed, depicted with the (unfilled or open symbols) 304, which does not affect the size distribution. The size distribution is very narrow, enabling a good control of the amount of drugs administrated to a patient.

After redispersion of the freeze dried capsules in a fluid medium, the capsules can be injected into a patient and the drug released by applying ultrasound energy using ultrasound imaging system 100. The drugs can be used for controlled release, for instance by an ultrasound pulse to effectuate local delivery. This is most efficient when targeted microspheres are used.

### EXAMPLE

12 □m particles were synthesized by inkjetting a solution of 0.1% of polylactic-acid, 0.05% of dodecane and 0.05% of paraffin containing 10% of a blue dye, oil blue N in dichloroethane into a 0.3% aqueous pva solution at the frequency of 25,000 Hz with the inkjet nozzle submerged in the solution. After washing 5 times the remaining dichloroethane was removed by evaporation and the particle size was measured using a Coulter counter and a modal diameter of 12 □m was found. The sample was freeze dried in two stages, 24 hours at 2 mbar followed by 24 hours at 0.03 mbar in the presence of glucose and polyethylene oxide. The particles were redispersed in water. The particles were subjected to ultrasound at a frequency of 1 MHz and an intensity of 2 W/cm². Release of the dye was observed by microscopy at 4000 frames per second.

Although the system and method of the present disclosure has been described with reference to exemplary embodiments thereof, the present disclosure is not limited to such exemplary embodiments. Rather, the system and method disclosed herein are susceptible to a variety of modifications, enhancements and/or variations, without departing from the spirit or scope hereof. Accordingly, the present disclosure embodies and encompasses such modifications, enhancements and/or variations within the scope of the claims appended hereto.

1. An in vivo polymeric drug delivery system, the system comprising:
therapeutic drug dissolved in a solvent, the solvent not being removable by lyophilization;
one of a gas and a gas precursor combined with the therapeutic drug dissolved in the solvent; and
a polymer shell, wherein the drug is released from within the polymer shell by application of ultrasound to rupture the polymer shell.

2. The system of claim 1, wherein the polymer shell is partially filled with the therapeutic drug dissolved in the solvent and partially filled with one of the gas and the gas precursor.

3. The system of claim 1, wherein both the solvent and gas precursor are liquids and one of the two liquids is phase converted using ultrasound.

4. The system of claim 1, wherein an inside surface defining the polymer shell is hydrophobic.

5. The system of claim 4, wherein the polymer shell is a polymer with one of an alkyl and a fluorinated end group.

6. The system of claim 1, wherein an outside surface defining the polymer shell includes a targeting moiety.

7. The system of claim 1, wherein the solvent is at least one of a higher alkane and an oil.

8. The system of claim 1, wherein the polymer shell is a biodegradable polymer including one of a polylactide, polyglycolide, polycaprolactone, polycyanoacrylate and copolymer of one of the foregoing.

9. The system of claim 8, wherein the biodegradable polymer is pegylated to improve circulation in blood.

10. The system of claim 1, wherein the system is substantially monodisperse

11. The system of claim 1, wherein the polymer shell is formed using drop-by-drop emulsification including one of inkjet printing, cross-flow emulsification and micro-channel emulsification.

12. A method for in vivo polymeric drug delivery using ultrasound, the method comprising:
dissolving a therapeutic drug in a solvent, the solvent not being removed by lyophilization;
combining one of a gas and a gas precursor with the therapeutic drug dissolved in a solvent, the gas and the gas precursor being removable by lyophilization;
forming a polymer shell by emulsification and lyophilization of the mixture of the therapeutic drug dissolved in the solvent combined with one of the gas and the gas precursor; and
applying ultrasound to rupture the polymer shell and release the drug in vivo.

13. The method of claim 12, wherein the polymer shell is partially filled with the therapeutic drug dissolved in the solvent and partially filled with one of the gas and the gas precursor.

14. The method of claim 12, wherein both the solvent and gas precursor are liquids and one of the two liquids is phase converted using ultrasound.

15. The method of claim 12, wherein an inside surface defining the polymer shell is hydrophobic.

16. The method of claim 15, wherein the polymer shell is a polymer with one of an alkyl and a fluorinated end group.

17. The method of claim 12, wherein an outside surface defining the polymer shell includes a targeting moiety.

18. The method of claim 12, wherein the solvent is at least one of a higher alkane and an oil.

19. The method of claim 12, wherein the polymer shell is a biodegradable polymer including one of a polylactide, polyglycolide, polycaprolactone, polycyanoacrylate and copolymer of one of the foregoing.

20. The method of claim 19, wherein the biodegradable polymer is pegylated to improve circulation in blood.

21. The method of claim 12, further comprising
forming substantially monodisperse particles.

22. The method of claim 12, further comprising:
forming the polymer shell using drop-by-drop emulsification including one of inkjet printing, cross-flow emulsification and micro-channel emulsification.

23. A method for polymeric drug delivery activated by ultrasound, the method comprising:
preparing an emulsion of at least one polymer dissolved in a first solvent and a drug dissolved in a second solvent;
adding a non-solvent;
removing solvent from the emulsion by one of agitation and extraction leaving a liquid consisting of non-solvent for the polymer to be evaporated and solvent for the drug;
redispersing the capsules in a fluid medium;
injecting the fluid medium having the capsules in vivo; and
applying ultrasound to release the drug from the capsule.

24. The method of claim 23, further comprising:
freeze drying the capsules at a selected pressure not sufficient to remove the solvent in which the drug is dissolved.
